(19) United States
(12) Patent Application Publication
(10) Pub. No.: US 2009/0012366 A1
(43) Pub. Date: Jan. 8, 2009

(54) RIGID-TYPE ELECTRONIC VIDEOENDOSCOPE

(76) Inventors: Soo Geun Wang, Busan (KR); Gye Rok Jeon, Busan (KR); Jung Hoon Ro, Busan (KR); Jeong Hwan Ok, Gyeonggi-do (KR); Yong Jin Wang, Busan (KR)

Correspondence Address: THE WEBB LAW FIRM, P.C., 700 KOPPERS BUILDING, 436 SEVENTH AVENUE, PITTSBURGH, PA, 15219, US

(21) Appl. No.: 11/628,147

(22) PCT Filed: Mar. 28, 2005
(86) PCT No.: PCT/KR2005/000905

§ 371 Date: Aug. 27, 2008

(30) Foreign Application Priority Data
May 31, 2004 (KR) 20-2004-0015145

(51) Int. Cl. A61B 1/06

(52) U.S. Cl. 600/178

(57) ABSTRACT

Disclosed herein is a rigid-type videoendoscope, which can allow a doctor to easily examine a patient's affected part using an image capturing part mounted on an end portion of a support tube, which can easily turn on and off LEDs for emitting light for allowing a CCD or CMOS image sensor to capture an image of the patient's affected part, and which can reduce electric power consumption, thereby improving economical efficiency.

### TECHNICAL FIELD

The present invention relates to a rigid-type electronic videoendoscope, which can allow a doctor to easily and selectively turn on and off an image capturing part mounted on a support tube inserted into the interior of a patient's body cavity and having LEDs for the visual examination of the lesion of the body cavity.

### BACKGROUND ART

Recently, due to the remarkable economic growth and rapid scientific development, the life quality of the moderns has been improved, and healthy life is the best target in life. Furthermore, people are highly concerned about health since they meet lots of information about health and diseases every day through mass media such as newspaper and broadcasting.

The reason is that the moderns are concerned about health since they recognize that health is threatened by mental stress increased due to rapidly scientific change and development and considerable increase of economic activities, by lack of exercise and adaptation to inadequate environment due to living in a restricted space, by air pollution due to rapid increase of cars, and so on.

Furthermore, in spite of such concerns, people suffer from various diseases. So, an endoscope is used for curing a patient's affected part. To examine the patient's affected part through the endoscope, a light source emits light to the interior of the patient's body cavity, a CCD camera captures an image of the patient's affected part lightened by the light source, and then, the captured data is displayed on a monitor, so that a doctor can easily examine and treat the patient's affected part.

However, in such conventional endoscope, since the light source generates heat severely, it cannot be directly mounted on the endoscope, and so is mounted separately. In addition, an optical fiber is used for inducing light to the patient's affected part. So, the conventional endoscope is complicated in structure, and expensive in manufacturing cost, and causes an economic loss since it needs an expensive CCD camera system for capturing an image of the patient's affected part.

### DISCLOSURE OF INVENTION

### Technical Solution

Accordingly, the present invention has been made in view of the above problems occurring in the prior art, and it is an object of the present invention to provide a rigid-type electronic videoendoscope, which can allow a doctor to easily examine a patient's affected part using an image capturing part mounted on an end portion of a support tube, which can easily turn on and off LEDs directly mounted thereon as a light source for emitting light for allowing a CCD or CMOS image sensor to capture an image of the patient's affected part, the LEDs being similar to a newly developed white light and generating a little heat since it is small in electric power consumption, and which is economical since it does not use an optical fiber which is expensive and needs optical inducement.

To achieve the above object, the present invention provides a rigid-type electronic videoendoscope including: a support tube inserted into the interior of a patient's body cavity for the visual examination of the lesion of the body cavity; an image capturing part having a housing mounted on the outer periphery of an end of the support tube, the housing having a guide rib protruding therefrom, a shielding plate mounted on the guide rib of the housing for shielding the inside and the outside of the housing, a CCD or CMOS image sensor mounted at the center of the shielding plate for capturing an image of a patient's affected part, and LEDs (Light Emitting Diode) radially mounted around the CCD or CMOS image sensor for emitting light; and a grip for receiving and supporting the other end of the support tube, the grip having operating buttons mounted thereon for operating the image capturing part.

### BRIEF DESCRIPTION OF THE DRAWINGS

FIG. 1 is a perspective view of an electronic videoendoscope according to the present invention.

FIG. 2 is an exploded perspective view of an image capturing part of the electronic videoendoscope according to the present invention.

FIG. 3 is a sectional view of the electronic videoendoscope according to the present invention.

### BEST MODE FOR CARRYING OUT THE INVENTION

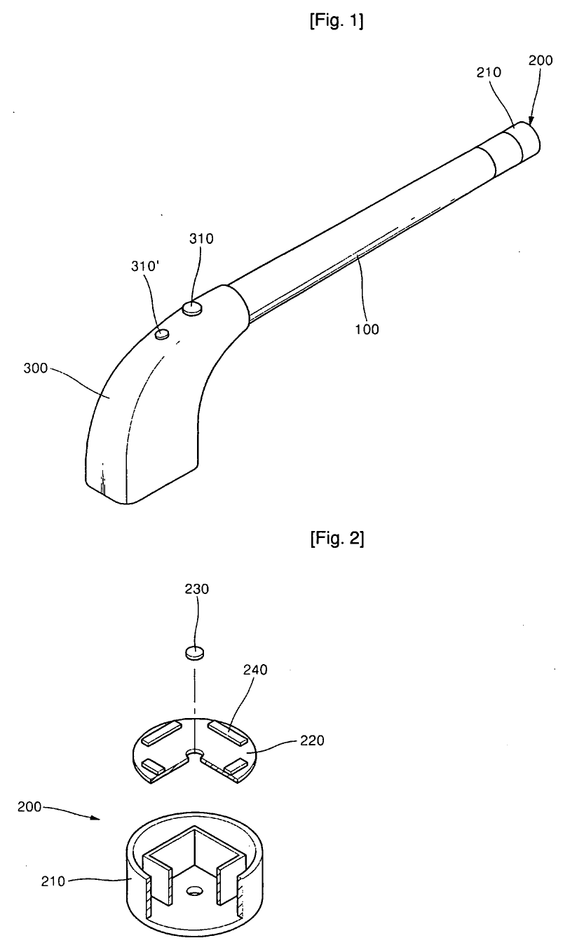

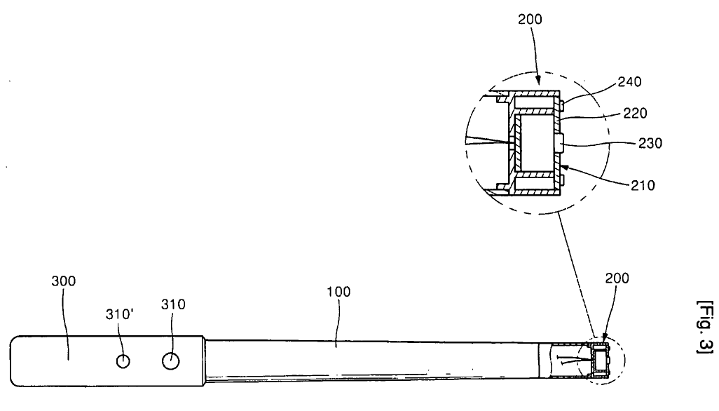

Reference will now be made in detail to the preferred embodiments of the present invention, examples of which are illustrated in the accompanying drawings. FIG. 1 is a perspective view of an electronic videoendoscope according to the present invention, FIG. 2 is an exploded perspective view of an image capturing part of the electronic videoendoscope, and FIG. 3 is a sectional view of the electronic videoendoscope.

The electronic videoendoscope according to the present invention includes a support tube 100, a shielding plate 220 and a grip 300.

The support tube 100 is inserted into the interior of a patient's body cavity for the visual examination of the lesion of the body cavity.

The image capturing part 200 comprises a housing 210, a shielding plate 220, a CCD or CMOS image sensor 230 and LEDs (Light Emitting Diode) 240. The housing 210 is mounted on an end of the support tube 100, and the housing 210 has a guide rib 211 protruding from the outer periphery of the housing 210. The shielding plate 220 is mounted on the guide rib 211 of the housing 210 for shielding the inside and the outside of the housing. The CCD or CMOS image sensor 230 is mounted at the center of the shielding plate 220 for capturing an image of a patient's affected part, and the LEDs (Light Emitting Diode) 240 are radially mounted around the CCD or CMOS image sensor 230 for emitting light.

The grip 300 receives and supports the other end of the support tube 100, and the grip 300 has operating buttons 310 and 310′ mounted thereon for operating the image capturing part 200.

Hereinafter, an assembly process of the present invention will be described. First, the CCD or CMOS image sensor 230 is mounted at the center of the shielding plate 220, and then, the LEDs 240 are radially mounted on the shielding plate 220 around the CCD or CMOS image sensor 230. After that, the shielding plate 220 is seated on the guide rib 211 of the housing 210, so that the image capturing part 200 is completely assembled.

At this time, electric wires have to be connected to the CCD or CMOS image sensor 230 and the LEDs 240 through the inside of the housing 210 for supplying electric power.

The electric wires protruding to the outside of the completely assembled image capturing part 200 is transferred to the inside of the support tube 100, and then, the image capturing part 200 is seated and fixed on the end portion of the support tube 100.

Furthermore, the On-Off operating buttons 310 and 310′ are mounted on the grip 300 for selectively supplying electric power to the electric wires.

Next, a use process of the present invention will be described.

First, a doctor grasps the grip 300 of the endoscope to examine the lesion of the patient's affected part, and presses the operating button 310 in order to selectively supply or cut off electric power to the LEDs 240. In a state where the LEDs 240 are supplied with electric power and turned on, when the doctor inserts the image capturing part 200 into the patient's body cavity, the LEDs 240 emit light at the end portion of the support tube 100 to throw light on the patient's affected part. Then, the doctor can examine the patient's affected part displayed on a monitor (not shown) through the CCD or CMOS image sensor 230. After that, the doctor presses the operating button 310′ to store an image of the patient's affected part, and then, can counsel with the patient about the patient's affected part and diagnose and treat the patient's affected part while seeing the stored data.

Moreover, when the doctor rotates the grip 300 in order to examine different position of the affected part, the image capturing part 200 is rotated, and the CCD or CMOS image sensor 230 captures an image of the patient's affected part lightened by the light of the LEDs 240, and then, the captured image is displayed on the monitor, so that the doctor can examine and treat the patient's affected part.

Meanwhile, when the doctor finishes the examination of the patient's affected part, the doctor presses the operating button 310 of the grip 300 to turn off the LEDs 240, and then, removes the endoscope from the patient's body cavity. The doctor disinfects the endoscope, and then, waits the next action.

The present invention can be widely used for dental surgery, opthalmology, dermatology, otorhinolaryngology, obstetrics and gynecology, and so on.

### INDUSTRIAL APPLICABILITY

As described above, the electronic endoscope according to the present invention can allow a doctor to easily examine a patient's affected part using an image capturing part mounted on an end portion of a support tube, and easily turn on and off LEDs directly mounted thereon as a light source for emitting light for allowing a CCD or CMOS image sensor to capture an image of the patient's affected part, the LEDs being similar to a newly developed white light and generating a little heat since it is small in electric power consumption. In addition, the electronic endoscope according to the present invention is economical since it does not use an optical fiber which is expensive and needs optical inducement.

1. A rigid-type electronic videoendoscope for allowing a doctor to examine the lesion of a patient's body cavity, comprising:
a support tube (100) inserted into the interior of a patient's body cavity for the visual examination of the lesion of the body cavity;
an image capturing part (200) having a housing (210) mounted on the outer periphery of an end of the support tube (100), the housing having a guide rib (211) protruding therefrom, a shielding plate (220) mounted on the guide rib (211) of the housing (210) for shielding the inside and the outside of the housing, a CCD or CMOS image sensor (230) mounted at the center of the shielding plate (220) for capturing an image of a patient's affected part, and LEDs (Light Emitting Diode) (240) radially mounted around the CCD or CMOS image sensor (230) for emitting light; and
a grip (300) for receiving and supporting the other end of the support tube (100), the grip having operating buttons (310)(310′) mounted thereon for operating the image capturing part (200).
wherein the grip (300) is bent downward for a user to observe the patient standing or sitting in front of the user, and the operating buttons (310)(310′) are mounted on an upper surface of the grip to secure the user's view.
